(19) United States
(12) Patent Application Publication
(10) Pub. No.: US 2003/0013078 A1
(43) Pub. Date: Jan. 16, 2003

(54) HIV-1 reporter viruses and their use in assaying anti-viral compounds

(76) Inventors: Wade Blair, San Marcos, CA (US); Timothy P. Spicer, Wethersfield, CT (US)

Correspondence Address: MORGAN & FINNEGAN, L.L.P., 345 Park Avenue, New York, NY, 10154-0053, US

(21) Appl. No.: 09/876,680

(22) Filed: Jun. 7, 2001

Related U.S. Application Data
(60) Provisional application No. 60/210,626, filed on Jun. 9, 2000.

(51) Int. Cl. C12N 15/867; C12N 7/01; C12Q 1/70; C12P 19/34; A61K 39/12; C12N 7/00; C12N 15/00; C12N 15/09; C12N 15/63; C12N 15/70; C12N 15/74; C12N 15/86

(52) U.S. Cl. 435/5; 435/456; 435/235.1; 435/320.1; 424/199.1; 435/91.1

(57) ABSTRACT

The present invention relates to HIV-1 proviral constructs, which produce replication competent HIV-1 reporter viruses. The invention also relates to the use of these HIV-1 reporter viruses in high throughput screening of anti-viral compounds.

### FIELD OF THE INVENTION

The present invention relates to replication competent HIV-1 proviral constructs that encode a reporter gene, preferably either the renilla luciferase or the secreted placental alkaline phosphatase (SEAP) reporter gene. The invention further relates to the use of these proviral construct virions in high throughput screening of compounds that inhibit HIV-1 replication.

### BACKGROUND OF THE INVENTION

Replication of the type 1 human immunodeficiency virus (HIV-1) in cell culture is commonly measured by quantitating the production of viral reverse transcriptase (RT) activity, quantitating mature viral capsid proteins (i.e., p24) in the cell supernatant, or by quantitating viral RNA copy number by reverse transcriptase directed polymerase chain reaction methods (RT-PCR). Alternatively, HIV-1 replication can be measured indirectly by quantitating viral induced cytopathic effect (cpe) on the infected cells using dye reduction methods such as XTT and MTT (Weislow et al, 1989). In the case of HIV-1 RT or p24 assays, the cumbersome nature of the methods exclude these type of analyses for high throughput anti-viral assays. Analysis of HIV replication by dye reduction methods such as XTT, on the other hand, involves a single step process and thus allows for higher throughput. However, as dye reduction methods are often performed after the majority of cells in the culture have been infected and killed, they may result in a reduction in sensitivity in the identification of less efficacious HIV-1 inhibitors. In addition, as dye reduction methods rely on significant cytopathic effect, they can not be used to accurately measure the replication of HIV strains that do not exhibit aggressive replication kinetics in cultured cell lines, such as non-syncitium inducing HIV-1 strains.

The generation of HIV-1 reporter viruses afforded an alternative to the traditional methods of measuring HIV-1 replication. Single-cycle infectious HIV-1 reporter viruses encoding luciferase as the reporter gene have been described (Chen et al., 1994). Those viruses allow simple and rapid analysis of approximately the first half of the HIV-1 replication cycle, including viral entry, reverse transcription, integration, and gene expression. However, steps post HIV gene expression in the infected cell, such as HIV protease mediated processing of viral precursor polypeptides required for virion maturation, are not easily measured using those viruses. In order to analyze the full replication cycle of HIV and to follow HIV-1 dissemination through a population of cells, replication competent reporter viruses are required. Several replication competent HIV-1 reporter viruses that can be used for the analysis of HIV-1 replication have been generated (Terwilliger, et al., 1989; Malim et al. 1992; He & Landau, 1995; Planelles, et al., 1995; Chen et al., 1996; Page et al., 1997; Jamieson and Zack, 1998). However, those reporter viruses are not useful for high volume anti-viral assays because the reporter gene products they encode cannot be measured by simple and rapid assays. For example, the expression of the chloramphenicol acetyl transferase (CAT) reporter gene can only be measured by cumbersome enzymatic assays with a limited range of sensitivity. Similarly, reporter viruses that encode a marker gene require fluorescence-activated cell sorting to identify HIV infected cells.

### SUMMARY OF THE INVENTION

The present invention relates to HIV-1 proviruses, which produce replication competent HIV-1 reporter viruses. These proviruses encode an HIV-1 genome in which a region of the genome that is not essential to viral replication has been substituted by a reporter gene, preferably, the renilla luciferase, a derivative of the renilla luciferase gene or the secreted placental alkaline phosphatase (SEAP) reporter gene. Derivatives of the renilla luciferase gene include amino acid or nucleotide substitutions in the wild type Renilla luciferase sequence (i.e. cys-ala substitutions) that result in a functional renilla luciferase enzyme. (Liu, et al., (1999)). The expression of the reporter gene is dependent on bonifide viral replication and can be detected using a simple and rapid assay.

The replication competent HIV-1 proviruses of the invention may be constructed using any replication competent HIV-1 proviral clones derived from any HIV-1 viral strain. In one embodiment, a replication competent HIV-1 provirus is constructed by replacing the nef gene of the HIV-1 proviral clone pNL4-3 with the renilla luciferase reporter gene. The resulting provirus produces T-tropic viruses that utilize CXCR4 as the co-receptor for viral infection. Other examples of reporter genes that can be used include, but are not limited to, the SEAP gene, the CAT gene or the green fluorescence protein gene (GFP).

In another embodiment, a replication competent HIV-1 provirus is constructed by replacing the nef gene of the HIV-1 proviral clone pNL4-3 with the renilla luciferase reporter gene, and replacing the envelope gene of pNL4-3 with the envelope gene of the HIV-1 provirus JRFL. The resulting provirus produces macrophage-tropic viruses that utilize CCR5 as the co-receptor for viral infection.

In yet another embodiment, a replication competent HIV-1 provirus is produced by replacing the nef gene of the HIV-1 proviral clone pNL4-3 with the SEAP reporter gene, and the envelope gene of pNL4-3 with the envelope gene of the HIV-1 proviral clone JRFL. The resulting provirus, designated JRFNSEAP, produces macrophage-tropic viruses that utilize CCR5 as the co-receptor for viral infection.

Other examples of envelope genes that can be used for generating the HIV-1 provirus of the invention include, but are not limited to, HIV-1 envelopes that display tropism for any of the CC or CXC chemokine receptors or any HIV-1 isolate envelope that is capable of initiating infection of a eukaryotic cell or cells.

The invention also relates to the use of the HIV-1 replication competent reporter viruses of the invention in high throughput in vitro screening of compounds that inhibit HIV-1 replication.

### BRIEF DESCRIPTION OF THE FIGURES

FIG. 1 is a schematic representation of the NL4RLuc proviral construct.

FIG. 2 is a schematic representation of the JRFNRLuc proviral construct.

FIG. 3 is a schematic representation of the JRFNFLuc proviral construct.

FIG. 4 is a schematic representation of the JRFNSEAP proviral construct.

FIG. 5 shows the kinetics of replication of HIV-1 viruses produced by the HIV-1 provirus pNL4RLuc. Renilla luciferase activity and the level of mature viral capsid protein p24 measured on days 0, 2 and 5 are shown.

FIG. 6 shows the kinetics of replication of HIV-1 viruses produced by the HIV-l provirus JRFNRluc. Renilla luciferase activity and the level of mature viral capsid protein p24 measured on days 0, 3 and 6 are shown.

FIG. 7 shows that HIV-1 virus produced following pNL4Rluc infection of MT-2 #18 cells were capable of initiating new rounds of infection in MT-2 #18 cells.

FIG. 8 shows that HIV-1 virus produced following JRFNRluc infection of MT-2 #18 cells were capable of initiating new rounds of infection in MT-2 #18 cells.

FIG. 9 shows the kinetics of replication of HIV-1 viruses produced by the HIV-1 provirus JRFNFLuc.

FIG. 10 shows the kinetics of replication of HIV-1 viruses produced by the HIV-1 provirus JRFNSEAP. SEAP activity and the level of mature viral capsid protein p24 measured on days 0, 3 and 6 are shown.

FIG. 11 shows that HIV-1 virus produced following JRFNSEAP infection of MT-2 #18 cells were capable of initiating new rounds of infection in MT-2 #18 cells.

FIG. 12 shows the p24 activity of the various HIV-1 reporter viruses and the pNL4-3 virus.

### DETAILED DESCRIPTION OF THE INVENTION

The present invention relates to replication competent HIV-1 proviruses. These replication competent HIV-1 proviruses are produced by replacing a region of an HIV-1 viral genome that is not essential to viral replication with a reporter gene, preferably the renilla luciferase gene, a derivative of the renilla luciferase gene or the secreted placental alkaline phosphatase (SEAP) reporter gene. Derivatives of the renilla luciferase gene include amino acid or nucleotide substitutions in the wild type Renilla luciferase sequence (i.e. cys-ala substitutions) that result in a functional renilla luciferase enzyme. (Liu, et al., (1999)). The expression of the reporter gene is dependent on bonifide viral replication and therefore serves as a method for quantifying viral replication. The expression of the reporter gene can be detected using a simple and rapid assay.

To construct the replication competent HIV-1 proviruses of the invention, a non-debilitating reporter gene, for example the renilla luciferase reporter gene, can be inserted anywhere in the HIV-1 viral genome so long as it does not disrupt the replication of the virus. Preferably, the reporter gene is inserted into the HIV-1 genome by replacing a region of the viral genome that is not essential to viral replication. Examples of such regions include, but are not limited to, the nef gene, the vpr gene, and fragments of the nef gene and vpr gene. Fragments of the nef and vpr gene are generally 1 to 300 nucleotides in length.

The replication competent HIV-1 proviruses of the invention may be constructed using any replication competent HIV-1 proviral clones derived from any HIV-1 viral strain. Examples of such clones include, but are not limited to, pNL4-3 (Adachi, et al., 1986), pYU-2 (Lai et al., 1991), p89.6 (Collman et al., 1992) and HIV-1 Lai (Wain-Hobson et al., 1985).

In one embodiment, a replication competent HIV-1 provirus is produced by replacing the nef gene of the HIV-1 proviral clone pNL4-3 with the renilla luciferase reporter gene. The resulting provirus, designated NL4Rluc, produces T-tropic viruses that utilize CXCR4 as the co-receptor for viral infection.

In another embodiment, a replication competent HIV-1 provirus is produced by replacing the nef gene of the HIV-1 proviral clone pNL4-3 with the renilla luciferase reporter gene, and the envelope gene of pNL4-3 with the envelope gene of the HIV-1 proviral clone JRFL. The resulting provirus, designated JRFNRluc, produces macrophage-tropic viruses that utilize CCR5 as the co-receptor for viral infection.

In yet another embodiment, a replication competent HIV-1 provirus is produced by replacing the nef gene of the HIV-1 proviral clone pNL4-3 with the SEAP reporter gene, and the envelope gene of pNL4-3 with the envelope gene of the HIV-1 proviral clone JRFL. The resulting provirus, designated JRFNSEAP, produces macrophage-tropic viruses that utilize CCR5 as the co-receptor for viral infection.

The expression of the renilla luciferase reporter gene, derivatives of the renilla luciferase gene or the SEAP reporter gene in cells infected with the HIV-1 viruses of the invention is dependent on bonifide viral replication in cell culture and can be detected using methods known in the art. In one embodiment, renilla luciferase activity is measured by adding the renilla luciferase substrate coelenteramine (Promega Corp., Madison, Wis.) to cells infected with the provirus. As renilla luciferase converts coelenteramine to coelenteramide with the concomitant production of CO₂ and light, the renilla luciferase activity may be detected by light emission which can be quantified using a scintillation counter or a luminometer.

In another embodiment, the SEAP activity is measured by adding the SEAP substrate CSPD (Tropix PE Applied Systems, Bedford, Mass.), a luminescence enhancer, and a buffer system that inhibits endogenous non-placental alkaline phosphatase activity to the supernatants from infected cell cultures. Upon dephosphorylation of the substrate by SEAP, a metastable phenolate anion intermediate is formed which decomposes and emits light. The light emission can be measured utilizing a scintillation counter or a luminometer.

It is understood that other substrates of renilla luciferase or SEAP known in the art may be used to measure renilla luciferase activity or SEAP activity.

As the replication of the HIV-1 viruses of the invention in cell culture can be readily measured using simple and rapid assays, the invention also relates to the use of these HIV-1 viruses in high throughput screening of compounds that inhibit HIV-1 replication.

In one embodiment, the method of screening comprises adding a test compound to cells infected with the viral stock of a replication competent provirus of the invention and measuring the level of renilla luciferase or SEAP activity 2 to 7 days, preferably 6 days, following incubation. In another embodiment, viral stocks may be added to a test compound together with the cells to be infected.

Suitable cells or cell lines for use in the screening of anti-viral compounds include, but are not limited to, MT-2 cell, MT-2 #18 cells, PM-1, CEM SS and Jurkat cells.

The level of renilla luciferase or SEAP activity in cells treated with the test compound is compared to that of the infected control cells. A reduction in the level of renilla luciferase or SEAP activity in cells treated with the test compounds relative to the level in the control cells indicates that the test compound may have the ability to inhibit HIV-1 viral replication. Preferably, the reduction is between 50 to 90%, and more preferably, it is more than 90%.

Viral stocks of the HIV-1 viruses of the invention can be generated by methods well known to those skilled in the art. In one embodiment, viral stocks are prepared by transfecting a proviral clone of the invention into cell cultures and harvesting the supernatants of the transfected cells. Transfection may be done using methods known in the art such as calcium phosphate, electroporation or liposome mediated transfection. Examples of cells that may be used for transfection include, but are not limited to, HEK 293 cells, Jurkat cells and CEM cells.

By measuring the change in the level of renilla luciferase or SEAP activity in cells treated with the test compound, it is possible to determine the effectiveness of the test compound in inhibiting viral replication as well as the degree of the effectiveness.

The present invention will now be described by way of examples, which are meant to illustrate, but not limit, the scope of the invention.

### EXAMPLES

### Materials and Methods

Cell Lines and Cell Culture Conditions

The MT-2 cell line, a CD4+ human malignant T-cell line, obtained from the NIH AIDS Research and Reference Reagent Program (Bethesda, Md.), were propagated in Roswell Park Memorial Institute (RPMI) 1640 medium (Life Technologies, Gaithersburg, Md.) containing 10% fetal Bovine serum (FBS, Life Technologies). This cell line naturally expresses CD4 and CXCR4 and was transformed to express human CCR5, the macrophage tropic HIV-1 co-receptor. The transformed cell line was designated by their clonal population number as MT-2 #18 and was cultured as its parental cell line MT-2 with the addition of 0.5 mg/ml of geneticin (Life Technologies, Gaithersburg, Md.). MT-2 #18 cells are permissive to HIV-1 infection via use of either CXCR4 co-receptor for T-tropic viruses or HIV-1 infection via CCR5 co-receptor for macrophage-tropic viruses.

HEK 293-T is a human embyronic kidney cell line transformed with the SV-40 large T-antigen. HEK 293-T cells were propagated in Dulbecco's Modified Eagle Medium (DMEM) (Life Technologies, Gaithersburg, Md.) containing 10% fetal Bovine serum.

Construction of Reporter Viruses

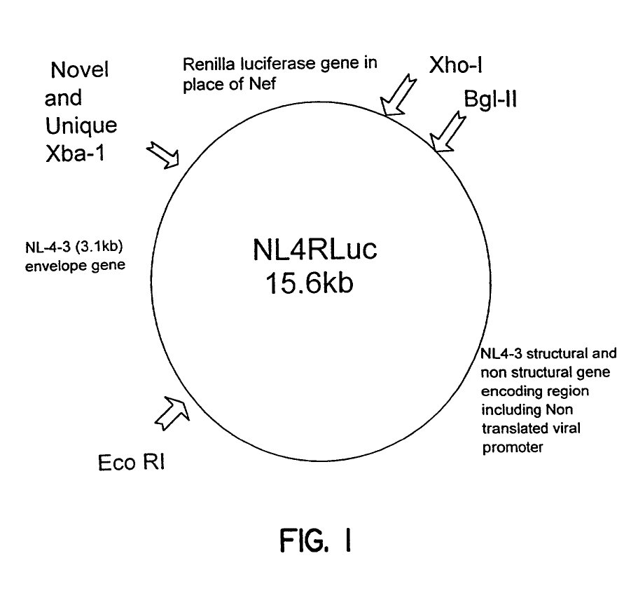

The HIV-1 proviral clone pNL4-3 (Adachi, et al., 1986) was used to construct the proviruses. To construct the NL4Rluc proviral clone (FIG. 1), a unique Xba I endonuclease restriction site was introduced immediately 3′ of the envelope coding sequences in pNL4-3. A 191 base pair segment between the introduced Xba I site and the Bgl II endonuclease restriction site in Nef were deleted and replaced by a unique Xho I endonuclease restriction site. The 947 base pair renilla luciferase reporter gene (RLuc) (Matthews et al., and Lorenz et al. was then inserted into the modified pNL4-3 using the introduced Xba I and Xho I sites. The resulting construct, NL4Rluc, is 756 base pairs longer than the pNL4-3 construct.

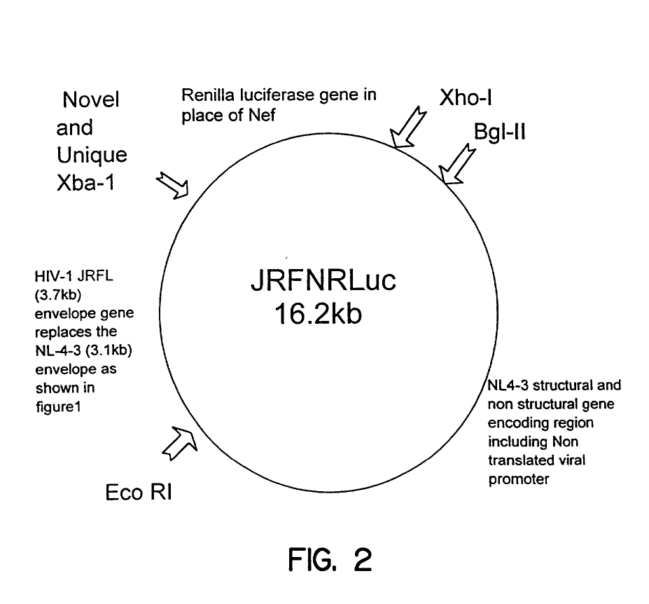

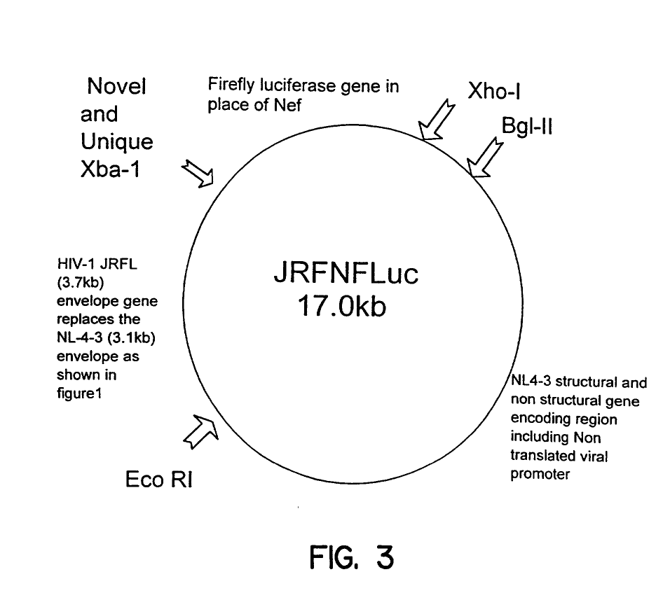

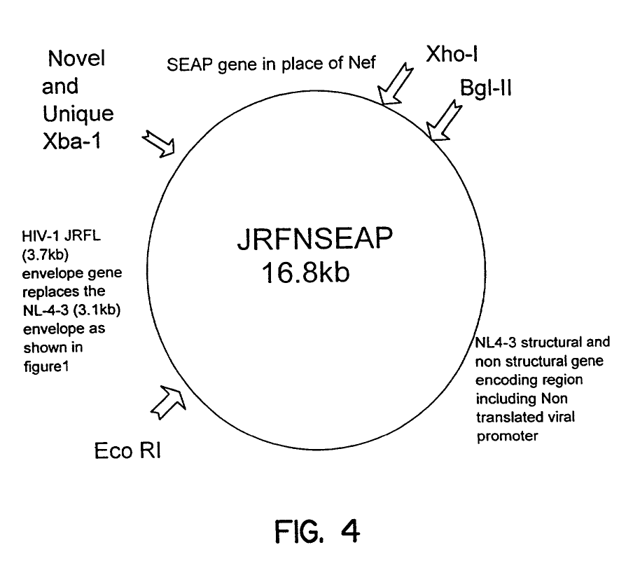

To construct the proviral clone JRFNRLuc (FIG. 2), the envelope-coding region of NL4Rluc (approximately 3,100 base pairs) was removed via the Eco RI site, located immediately 5′ to the envelope coding sequence, and the aforementioned unique Xba I site. This fragment was replaced by the envelope coding region of HIV-1 JRFL (approximately 3,700 base pairs) (O'Brien, et al., 1990). The envelope coding region of the JRFL clone was prepared utilizing PCR amplification and recombinant cloning of this region at the Eco RI and Xba I sites. The JRFNFLuc cDNA clone (FIG. 3) was constructed by replacing the renilla luciferase gene in JRFNRLuc (947 base pairs) with the firefly luciferase gene (1,706 base pairs). The proviral clone JRFNSEAP was constructed by replacing the renilla luciferase gene in JRFNRLuc with the SEAP encoding gene (1,557 base pairs) utilizing the Xba I and Xho I sites (FIG. 4).

Preparation of Virus Stocks

HEK 293 cells were transfected with the NL4Rluc, JRFNRLuc, JRFNFLuc or JRFNSEAP proviral clone using LipofectAMINE plus (Life Technologies, Gaithersburg, Md.) according to the manufacturer's instructions. Approximately 3×10⁷ cells were used in each transfection. Transfected cell supernatants were harvested 72 hours after transfection, clarified by low speed centrifugation, and stored at −70° C. Infectious virus was thawed and titered using MT-2 #18 cells.

Infection of Cells and p24 Assay

MT-2 #18 cells were pelleted and infected with virus stocks at a multiplicity of infection (MOI) of 0.01 at 37° C. for 2hrs. Viruses were removed and cells were washed twice with 1×PBS before re-suspending in a final concentration of 10,000 cells/100 uL. Infectivity and drug sensitivity assays were carried out in 96 well tissue culture plates, and kinetic studies were carried out in T-25 flasks. Cell supernatants were harvested on day 0, 2, 3, 5 or 6, and passaged virus was harvested on day 2 or 3. The activity for the capsid protein p24 was analyzed according to manufacturer's protocol utilizing the Zeptometrix Retrotek product (Zeptometrix Incorporated, Buffalo, N.Y.).

High Throughput Screening Assay

Large volume cultures of MT-2 #18 cells were prepared and kept at a concentration of less than 0.5×10⁶ cells per milliliter in RPMI media with 10% fetal calf serum. Test compounds were plated into a 96 well tissue culture treated clear bottom black plates (Corning Incorporated, Corning, N.Y.) at a concentration of 3 mM in 20 microliters of dionized water and 5% dimethyl sulfoxide. MT-2 #18 cells were pelleted via low speed centrifugation and re-suspended in fresh media. Reporter viruses were added to the fresh media and cells to obtain the appropriate MOI of 0.01 and a cell concentration of 1×10⁴ in 160 microliters. Twenty microliters per well of 2×complete modified eagle's media was then added to compensate for the dionized water volume. 180 microliters of this mixture is then added to each test compound well and control wells. Following a 5 to 6 day incubation at 37° C., media was aspirated from the wells utilizing a 12-channel manifold.

Renilla luciferase activity was measured using the Promega Dual Luciferase Reporter kit (Promega Corp., Madison, Wis.). Substrate was added to the plates directly onto the bare cells in each well utilizing a Multidrop instrument (Labsystems, Franklin, Mass.) in batches of ten. Following the substrate addition, plates were immediately sealed and read for 5 seconds per well in a Wallac Microbeta 1450 (Perkin-Elmer Wallac Incorporated, Gaithersburg, Md.) in the luminescence mode.

Secreted placental alkaline phosphatase (SEAP) activity was measured using the a 1,2 dioxetane alkaline phosphatase substrate called CSPD (Tropix PE Applied Biosystems, Bedford, Mass.). The assay includes two incubation steps including incubation at 65° C. for 5-30 minutes but, preferably 30 minutes, and then adding L-homoarginine for a period of 5-20 minutes, preferably 20 minutes. Light emission can be measured utilizing a scintillation counter or a luminometer.

### Example 1

Replication of Renilla Luciferase Reporter Viruses

The pNL4-3 construct, a well-characterized CXCR4-tropic HIV-1 proviral clone, was used as the proviral cDNA backbone for the construction of HIV-1 proviruses. Three proviral clones encoding the renilla luciferase gene, NL4Rluc, JRFNRluc, and FNFLuc, were constructed. The NL4Rluc clone was constructed by replacing the nef gene in the HIV-1 genome with the renilla luciferase reporter gene. The NL4Rluc progeny virions utilize CXCR-4 as the co-receptor for viral infection. The JRFNRluc clone was constructed by replacing the envelope region of the NL4Rluc clone with the envelope region of the HIV-1 strain JRFL. The JRFNFLuc clone was constructed by replacing the renilla luciferase reporter gene of the JRFNRluc clone with the firefly luciferase gene. The progeny virions of JRFNRluc and JRFNFLuc utilize CCR5 as the co-receptor for viral infection.

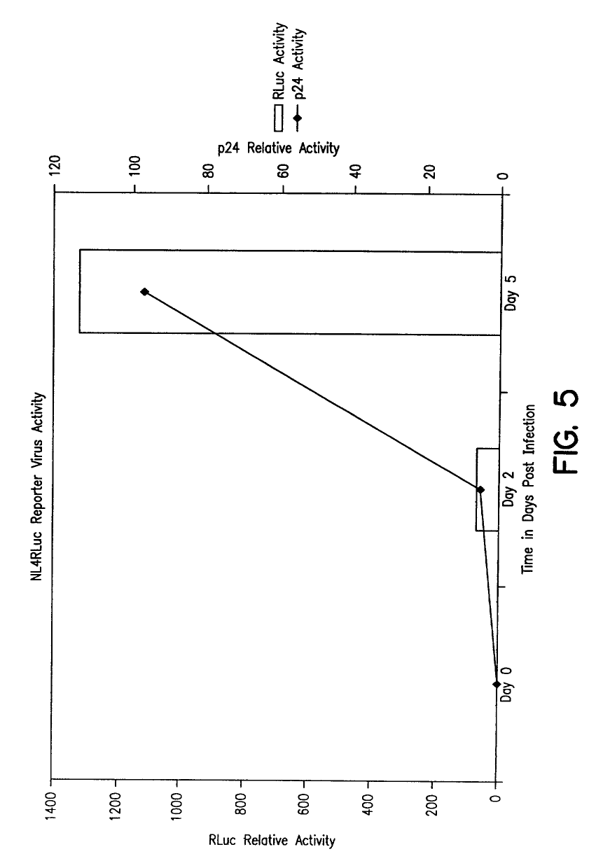

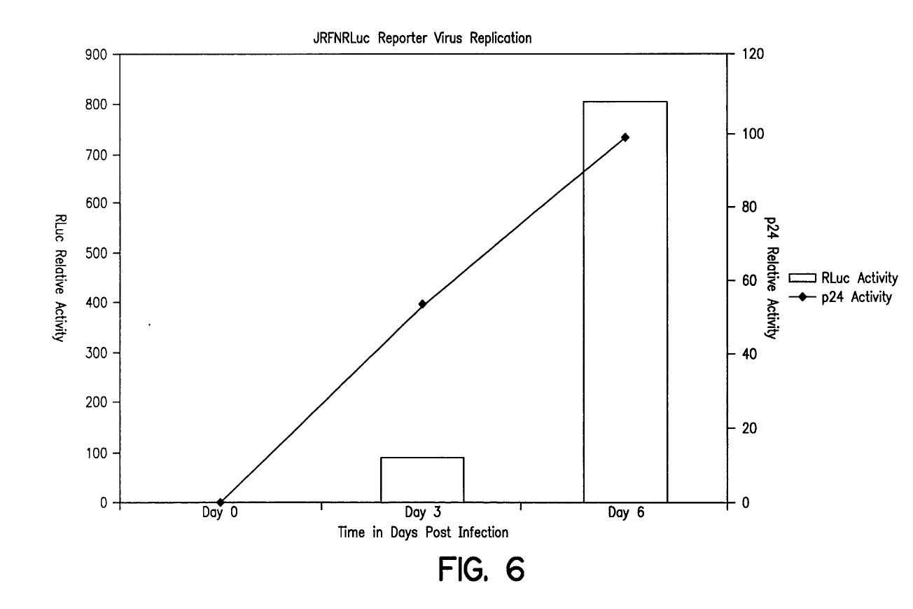

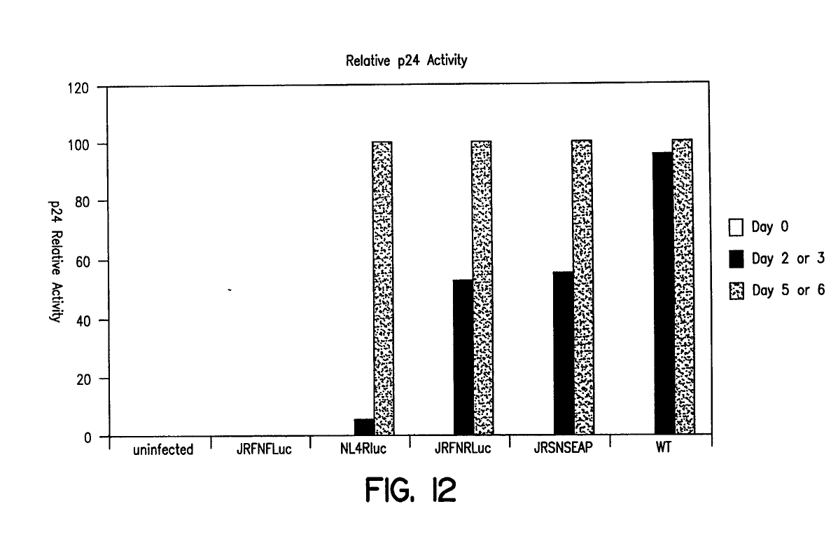

The ability of viruses derived from these clones to replicate in vitro was tested. Five days following infection, renilla luciferase activity in MT-2 #18 cells infected with NL4Rluc was more than 1000-fold over what was observed in uninfected cells (FIG. 5). Similarly, the production of renilla luciferase activity in MT-2 #18 cells infected with JRFNRLuc was more than 800-fold over what was observed in uninfected cells (FIG. 6). The kinetics of renilla luciferase activity in cells infected with either virus correlated with p24 (a capsid protein present in mature virions) production (FIGS. 5 and 6). This suggests that the renilla luciferase activity detected resulted from HIV-1 viral replication. The finding that the level of p24 production by the NL4Rluc and JRFNRluc viruses was virtually identical to that of the wild type pNL4-3 progeny virus demonstrates that replacement of the nef gene by the renilla luciferase gene had no detrimental effect on HIV-1 viral replication (FIG. 12).

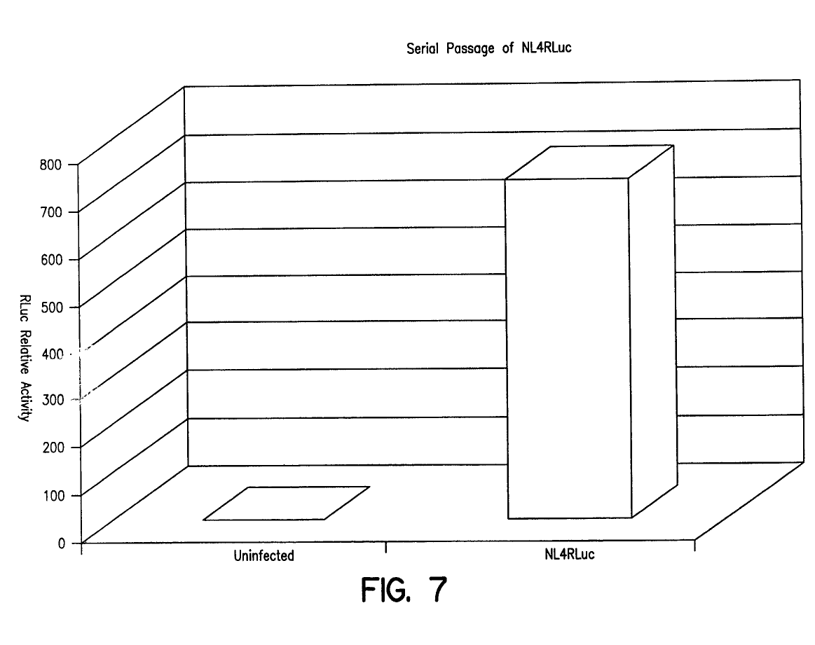

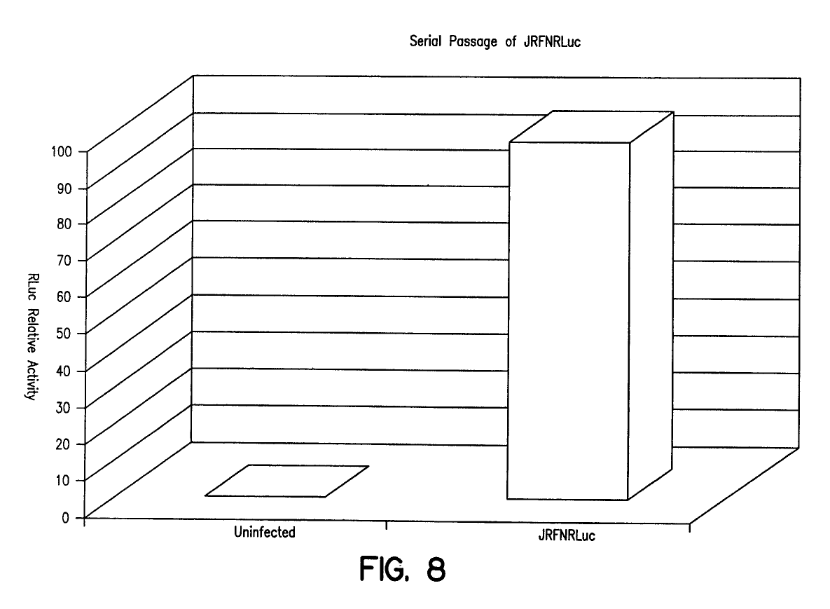

To determine whether the HIV-1 viruses produced following NL4Rluc and JRFNRLuc infection of cell cultures were capable of initiating new rounds of infection in cell culture, MT-2 #18 cells were infected by NL4Rluc or JRFNRLuc supernatants and p24 activity and renilla luciferase activity were measured. As shown in FIG. 7, NL4Rluc viruses produced after infection of T-cell lines for 6 days were capable of transducing the reporter gene to fresh MT-2 #18 cells. Likewise, JRFNRLuc virions produced after infection of T-cell lines for 6 days were capable of transducing the reporter gene to fresh MT-2 #18 cells (FIG. 8). These results demonstrate that the NL4Rluc and JRFNRLuc viruses are capable of being passed from infected cell cultures to initiate multiple rounds of replication in fresh cell cultures and hence, are replication competent.

The replication property of the NL4Rluc and JRFNRLuc viruses was studied using the HIV-1 protease inhibitor Amprenavir (Glaxo Wellcome, Research Triangle Park, N.C.) and the non-nucleoside reverse transcriptase inhibitor Efavirenz (Dupont Pharmaceuticals, Wilmington, Del.). Amprenavir and Efavirenz are clinically used for the intervention of HIV-1 infection. The reverse transcriptase inhibitor Efavirenz acts early in the HIV-1 replication cycle, whereas the protease inhibitor Amprenavir acts late in the HIV-1 replication cycle. As shown in Table 1, infection of MT-2 #18 cells by NL4Rluc and JRFNRLuc was inhibited by Efavirenz as well as by Amprenavir. Amprenavir inhibited NL4Rluc and JRFNRLuc, replication with an effective concentration 50 (EC 50) value of 19.4 nM and 6.9 nM, respectively, and Efavirenz inhibited NL4Rluc and JRFNRLuc replication at an EC50 of 0.36 nM and 0.12 nM, respectively. The HIV-1 protease inhibitor Amprenavir acts late in the replication cycle during virion maturation and would not inhibit a single cycle infection. Therefore, the results further demonstrate that the observed renilla luciferase reporter gene activity results from bona fide NL4Rluc or JRFNRLuc replication.

TABLE 1
Inhibition of Viral Replication
by Amprenavir and Efavirenz

| | Efavirenz | Amprenivir | |
|---|---|---|---|
| Provirus | EC50 | EC50 | |
| NL4RLuc | 0.36 nM | 19.4 | nM |
| JRFNRLuc | 0.12 nM | 6.9 | nM |
| JRFNFLuc | 0.18 nM | >10,000 | nM |
| JRFNSEAP | 0.10 nM | 21.5 | nM |

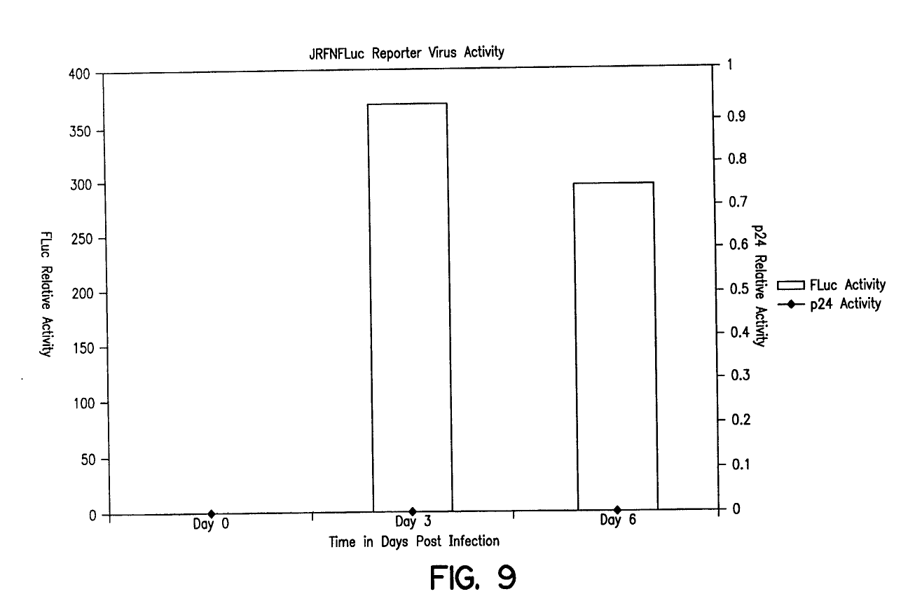

The replication ability of the NL4Rluc and JRFNRLuc viruses were then compared to that of the JRFNFLuc virus. JRFNFLuc differs from JRFNRLuc in that it encodes the firefly luciferase gene instead of the renilla luciferase gene. JRFNFLuc was constructed in a fashion similar to that previously reported (Chen et al., 1994). Infection of T-cell lines by JRFNFLuc was carried out exactly as that was done for NL4Rluc and JRFNRLuc viruses. As shown in FIG. 9, infection of cells with JRFNFLuc resulted in the production of firefly luciferase activity that was over 200-fold what was observed in uninfected cells 3 days after infection. However, the level of activity did not increase beyond this point in time and p24 activity never increased above background levels during the course of 6 days. The results indicate that mature virus core particles were not being made. Upon passing the day 6 post infection JRFNFLuc supernatant onto uninfected MT-2 #18 cells, no firefly luciferase activity or p24 production could be detected up to seven days post inoculation (data not shown). The results suggest that the supernatants did not contain virus capable of initiating new rounds of replication. Finally, the non-nucleoside reverse transcriptase inhibitor Efavirenz inhibited JRFNFLuc with an effective concentration 50 (EC 50) value of 0.18 nM but the HIV-1 protease inhibitor Amprenavir was unable to inhibit this virus at 10 uM; a concentration that is over 1000 fold that of the replication competent JRFNRLuc (Table 1). The finding that this virus was not capable of inhibition by a late stage HIV-1 replication cycle inhibitor is further evidence of it not being able to replicate. Taken together, these data strongly suggests that the JRFNFLuc virus is only capable of a single round of infection and is not replication competent, whereas NL4Rluc and JRFNRLuc are capable of multiple rounds of infection and are bona fide replication competent proviruses.

### Example 2

Replication of the SEAP Reporter Virus

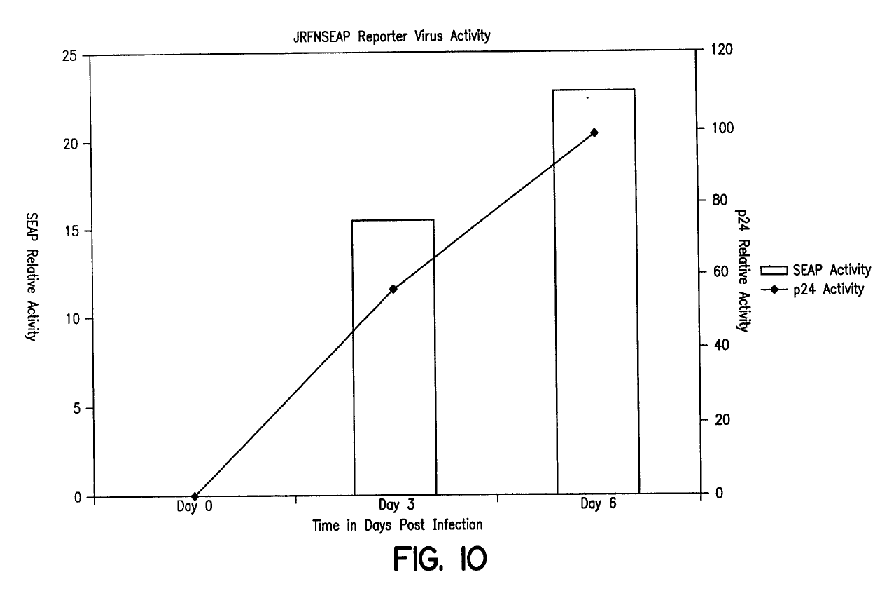

The JRFNSEAP proviral construct was made by replacing the renilla luciferase gene in the JRFNRLuc with the gene encoding SEAP. The ability of viruses produced by the proviral clone to replicate in vitro was tested. Six days following infection, SEAP activity in MT-2 #18 cells infected with JRFNSEAP was more than 150-fold greater than the activity observed in the uninfected cells. The kinetics of SEAP activity in cells infected with the virus correlated with p24 activity (FIG. 10). This suggests that the SEAP activity detected resulted from HIV-1 viral replication. The finding that the level of p24 production by the JRFNSEAP virus was virtually identical to that of the wild type pNL4-3 progeny virus demonstrates that replacement of the nef gene by the SEAP gene had no detrimental effect on HIV-1 viral replication (FIG. 12).

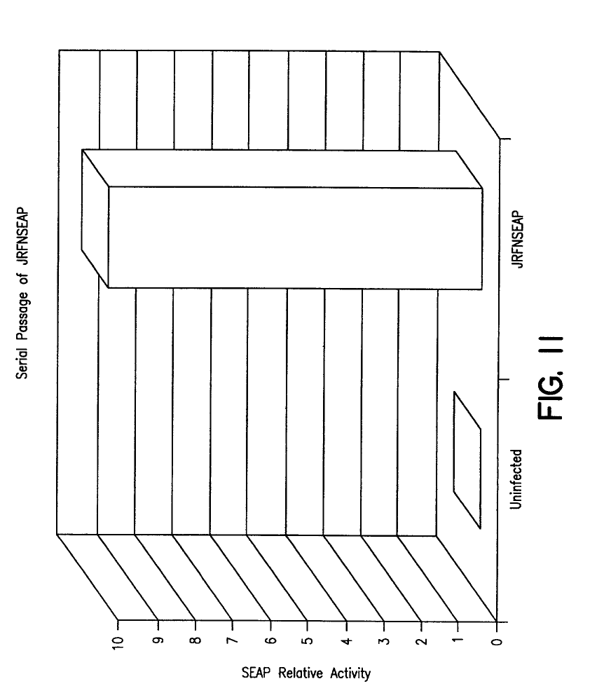

Experiments were then carried out to determine whether the JRFNSEAP virus produced following infection of cell cultures was capable of initiating new rounds of infection in cell culture. As shown in FIG. 11, JRFNSEAP virus produced after infection of T-cell lines for 6 days was capable of transducing the reporter gene to fresh MT-2 #18 cells. The results demonstrate that the SEAP reporter virus is capable of being passed from infected cell cultures to initiate multiple rounds of replication in fresh cell cultures and, hence, is replication competent.

The replication property of the JRFNSEAP virus was studied using the HIV-1 protease inhibitor Amprenavir and the non-nucleoside reverse transcriptase inhibitor Efavirenz as described above. As shown in Table 1, infection of MT-2 #18 cells by JRFNSEAP was inhibited by Efavirenz as well as by Amprenavir. Amprenavir inhibited JRFNSEAP replication with an effective concentration 50 (EC 50) value of 21.5 nM, and Efavirenz inhibited replication at an EC50 of 0.10 nM. The HIV-1 protease inhibitor Amprenavir acts late in the replication cycle during virion maturation and would not inhibit a single cycle infection. Therefore, the finding further demonstrates that the observed SEAP reporter gene activity results from bonifide viral replication.

References

1. Chen et al., 1994, J. Virol. 68:654-660.

2. Terwilliger, et al., 1989, PNAS 86:3857-3861.

3. Malim et al., 1992, J. Exp. Med. 176:1197-1201.

4. He & Landau, 1995, J. Virol. 69:4587-4592.

5. Planelles, et al., 1995, Gene Therapy 2:369-376.

6. Chen et al., 1996, J. Virol. 70:6044-6053.

7. Page et al., 1997, Aids Research and Human Retro. 13:1077-1081.

8. Jamieson and Zack, 1998, J. Virol.72: 6520-6526.

9. Weislow et al., 1989, J. of Nat.Cancer Inst. 81: 577-586.

10. Adachi et al., 1986, J. Virol. 59: 284-291.

11. O'Brien et al., 1990, Nature 348: 69-73.

12. Matthews et al., 1977, Biochemistry 16: 85.

13. Edinger et al., 1998, J. Virol. 72: 7934-7940.

14. Wain-Hobson et al., 1985, Cell 40: 9-17.

15. Li et al., 1991, J. Virol. 65: 3973-3985.

16. Collman et al., 1992, J. Virol. 66: 7517-7521.

17. Lorenz et al., 1991, P.N.A.S. USA 88: 4438.

18. Liu et al., 1999, Gene 237: 153-159.

The contents of all patents, patent applications, published articles, reference manuals, texts and abstracts cited herein are hereby incorporated by reference in their entirety to more fully describe the state of the art to which the present invention pertains.

As various changes can be made in the above compositions and methods without departing from the scope and spirit of the invention, it is intended that all subject matter contained in the above description, shown in the accompanying drawings, or defined in the appended claims be interpreted as illustrative, and not in a limiting sense.

1. A vector that encodes a replication competent HIV-1 virus, said vector comprising an HIV-1 genome in which a region non-essential for viral replication has been replaced by a reporter gene.

2. The vector according to claim 1 wherein said reporter gene is selected from the group consisting of the renilla luciferase reporter gene, the SEAP reporter gene, the CAT gene, and the green fluorescence protein gene.

3. The vector according to claim 2 wherein said reporter gene is selected from the group consisting of the renilla luciferase reporter gene and the SEAP reporter gene.

4. The vector according to claims 1, 2 or 3 wherein the region non-essential for viral replication encodes the nef gene or a fragment of the nef gene.

5. The vector according to claims 1, 2 or 3 wherein the region non-essential for viral replication encodes the vpr gene or a fragment of the vpr gene.

6. The vector according to claims 1, 2 or 3 wherein the HIV-1 genome is the genome of the pNL4-3 proviral clone.

7. The vector according to claims 1, 2 or 3 wherein the HIV-1 genome is the genome of the pYU-2 proviral clone.

8. The vector according to claims 1, 2 or 3 wherein the HIV-1 genome is the genome of the p89.6 proviral clone.

9. The vector according to claims 1, 2 or 3 wherein the HIV-1 genome is the genome of the HIV-1 Lai proviral clone.

10. A cell comprising the vector of claim 1, 2 or 3.

11. A method of screening for compounds that exhibit anti-viral activity against HIV-1 comprising:
a) adding a test compound to mammalian cells infected or cells to be infected with the vector according to claim 1, 2 or 3; and
b) comparing reporter gene activity in cells exposed to the test compound to the level of expression in control cells,
wherein a reduction in the level of reporter gene expression indicates the test compound inhibits HIV-1 replication.

12. The method according to claim 8, wherein the mammalian cells are MT-2 #18 cells.

13. A vector that encodes a replication competent HIV-1 virus, said vector comprising an HIV-1 genome in which a region non-essential for viral replication has been replaced by a nucleic acid sequence encoding a functional renilla luciferase enzyme.

14. The vector according to claim 13 wherein the renilla luciferase gene contains a cysteine to alanine substitution that results in a functional renilla luciferase enzyme.

15. The vector according to claim 13 wherein the region non-essential for viral replication encodes the nef gene or a fragment of the nef gene.

16. The vector according to claim 13 wherein the region non-essential for viral replication encodes the vpr gene or a fragment of the vpr gene.

17. The vector according to claim 13 wherein the HIV-1 genome is the genome of the pNL4-3 proviral clone.

18. The vector according to claim 13 wherein the HIV-1 genome is the genome of the pYU-2 proviral clone.

19. The vector according to claim 13 wherein the HIV-1 genome is the genome of the p89.6 proviral clone.

20. The vector according to claim 13 wherein the HIV-1 genome is the genome of the HIV-1 Lai proviral clone.

21. A cell comprising the vector of claim 13.

22. A method of screening for compounds that exhibit anti-viral activity against HIV-1 comprising:
a) adding a test compound to mammalian cells infected or cells that will be infected with the vector according to claim 13; and
b) comparing reporter gene activity in cells exposed to the test compound to the level of expression in control cells,
wherein a reduction in the level of reporter gene expression indicates the test compound inhibits HIV-1 replication.

23. The method according to claim 13, wherein the mammalian cells are MT-2 #18 cells.
